(19) United States
(12) Patent Application Publication
(10) Pub. No.: US 2011/0158987 A1
(43) Pub. Date: Jun. 30, 2011

(54) NOVEL ANTIBODY FORMULATION

(75) Inventors: Michael Adler, Riehen (CH); Ulla Grauschopf, Riehen (CH); Hanns-Christian Mahler, Basel (CH)

(73) Assignee: F. Hoffmann-LaRoche AG

(21) Appl. No.: 12/977,509

(22) Filed: Dec. 23, 2010

(30) Foreign Application Priority Data
Dec. 29, 2009 (EP) 09180840.2

(51) Int. Cl. A61K 39/395; A61P 35/00

(52) U.S. Cl. 424/133.1; 424/172.1; 424/143.1

(57) ABSTRACT

This invention relates to a pharmaceutical formulation of an antibody against Epidermal Growth Factor Receptor (EGFR), a process for the preparation and uses of the formulation.

This application claims priority to European Patent Application no. 09180840.2. filed Dec. 29, 2009, the contents of which is incorporated in its entirety herein by reference.

The present invention relates to a pharmaceutical formulation of an antibody against Epidermal Growth Factor Receptor (anti-EGFR antibody), a process for the preparation of said formulation and uses of the formulation.

In a first aspect, the invention relates to a pharmaceutical formulation comprising:

1 to 200 mg/ml of an IgG-class anti-EGFR antibody;
1 to 100 mM of a buffering agent;
0.001 to 1% (w/v) of a surfactant;
1 to 500 mM of at least one stabilizer;
at a pH in the range of from 4.0 to 7.0.

The formulation according to the invention may be provided in liquid form, lyophilized form or in liquid form reconstituted from a lyophilized form.

Unless otherwise defined in the following, terms are used herein as generally used in the art.

The term “IgG-class anti-EGFR antibody”, as used herein, includes antibodies of the immunoglobulin G (IgG) class of immunoglobulins, which target the human epidermal growth factor receptor (EGFR), also known as HER-1 or ErbB-1 (Ullrich et al., Nature 309, 418-425 (1984); SwissProt Accession #P00533; secondary accession numbers: O00688, O00732, P06268, Q14225, Q68GS5, Q92795, Q9BZS2, Q9GZX1, Q9H2C9, Q9H3C9, Q9UMD7, Q9UMD8, Q9UMG5), as well as naturally-occurring isoforms and variants thereof.

Exemplary IgG-class anti-EGFR antibodies useful in the formulation according to the present invention include cetuximab/IMC-C225 (Erbitux®, described in Goldstein et al., Clin Cancer Res 1, 1311-1318 (1995)), panitumumab/ABX-EGF (Vectibix®, described in Yang et al., Cancer Res 59, 1236-1243 (1999), Yang et al., Critical Reviews in Oncology/Hematology 38, 17-23 (2001)), nimotuzumab/h-R3 (TheraCim®, described in Mateo et al., Immunotechnology 3, 71-81 (1997); Crombet-Ramos et al., Int J Cancer 101, 567-575 (2002), Boland & Bebb, Expert Opin Biol Ther 9, 1199-1206 (2009)), matuzumab/EMD 72000 (described in Bier et al., Cancer Immunol Immunother 46, 167-173 (1998), Kim, Curr Opin Mol Ther 6, 96-103 (2004)), and zalutumumab/2F8 (described in Bleeker et al., J Immunol 173, 4699-4707 (2004), Lammerts van Bueren, PNAS105, 6109-6114 (2008)).

Particular IgG-class anti-EGFR antibodies useful in the formulation according to the present invention are described in WO 2006/082515 and WO 2008/017963, the entire content of which is incorporated herein by reference, and include antibodies which are characterized in that they are chimeric antibodies having the binding specificity of the rat monoclonal antibody ICR62 and that their effector functions are enhanced by altered glycosylation.

Particular antibodies are characterized in that they comprise at least one (i.e. one, two, three, four, five, or six) complementarity determining region (CDR) of the rat ICR62 antibody, or a variant or truncated form thereof containing at least the specificity-determining residues for said CDR, and comprising a sequence derived from a heterologous polypeptide. By “specificity-determining residue” is meant those residues that are directly involved in the interaction with the antigen. Specifically, particular antibodies comprise: (a) a heavy chain CDR1 sequence selected from a group consisting of: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, and SEQ ID NO:13; (b) a heavy chain CDR2 sequence selected from a group consisting of: SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30; and (c) the heavy chain CDR3 sequence SEQ ID NO:31. Particular antibodies further comprise: (a) a light chain CDR1 sequence selected from the group consisting of SEQ ID NO:32 and SEQ ID NO:33; (b) the light chain CDR2 sequence SEQ ID NO:34; and (c) the light chain CDR3 sequence SEQ ID NO:35.

In a particular embodiment, antibodies are characterized in that they comprise at least three CDRs of the rat ICR62 antibody, or variants or truncated forms thereof containing at least the specificity-determining residues for said CDRs.

In a particular embodiment, antibodies comprise:

a) in the heavy chain variable domain a CDR1 of SEQ ID NO:1, a CDR2 of SEQ ID NO:16, and a CDR3 of SEQ ID NO:31, and
b) in the light chain variable domain a CDR1 of SEQ ID NO:33, a CDR2 of SEQ ID NO:34, and a CDR3 of SEQ ID NO:35.

In a particular embodiment, antibodies comprise the heavy chain variable domain of the rat ICR62 antibody according to SEQ ID NO:36, or a variant thereof; and a non-murine polypeptide. Further, particular antibodies may comprise the light chain variable domain of the rat ICR62 antibody according to SEQ ID NO:37, or a variant thereof; and a non-murine polypeptide.

In a particular embodiment, antibodies comprise the heavy chain variable domain of SEQ ID NO:38 and the light chain variable domain of SEQ ID NO:39.

In a particular embodiment, antibodies are primatized or, in another particular embodiment, humanized antibodies.

A “humanized” antibody refers to a chimeric antibody comprising amino acid residues from non-human hypervariable regions (HVRs) and amino acid residues from human framework regions (FRs). A humanized antibody comprises substantially typically two variable domains, in which all or substantially all of the HVRs (e.g., complementarity determining regions (CDRs)) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Humanization may be achieved by various methods known in the art, including, but not limited to, (a) grafting the entire non-human variable domains onto human constant regions to generate chimeric antibodies, (b) grafting only the non-human (e.g., donor antibody) CDRs onto human (e.g., recipient antibody) framework and constant regions with or without retention of critical framework residues (e.g., those that are important for retaining good antigen binding affinity or antibody functions), (c) grafting only the non-human specificity-determining regions (SDRs or a-CDRs; the residues critical for the antibody-antigen interaction) onto human framework and constant regions, or (d) transplanting the entire non-human variable domains, but “cloaking” them with a human-like section by replacement of surface residues.

In a particular embodiment, the antibodies useful in the formulation according to the present invention comprise a human Fc region. In a particular embodiment, the human heavy chain constant region is Ig gamma-1, as set forth in SEQ ID NO:40, i.e. the antibody is of human IgG1 subclass.

In a particular embodiment, antibodies have been glycoengineered to have an altered oligosaccharide structure in the Fc region.

Specifically, particular antibodies have an increased proportion of non-fucosylated oligosaccharides in the Fc region as compared to non-glycoengineered antibodies. In a particular embodiment, the percentage of non-fucosylated oligosaccharides is at least 20%, at least 50, at least 70%, or at least 75%. The non-fucosylated oligosaccharides may be of the hybrid or complex type.

In a particular embodiment, antibodies may also have an increased proportion of bisected oligosaccharides in the Fc region. In a particular embodiment, the percentage of bisected oligosaccharides in the Fc region of the antibody is at least 50%, at least 60%, at least 70%, at least 80%, at least 90%, or at least 90-95% of the total oligosaccharides. In a particular embodiment, antibodies have an increased proportion of bisected, non-fucosylated oligosaccharides in the Fc region. The bisected, non-fucosylated oligosaccharides may be either hybrid or complex. In particular embodiment, at least 15%, at least 20%, at least 25%, at least 30% or at least 35% of the oligosaccharides in the Fc region of the antibody are bisected, non-fucosylated.

The term “glycoengineered”, as used herein, includes any manipulation of the glycosylation pattern of a naturally occurring or recombinant protein, polypeptide or a fragment thereof. Glycoengineering includes metabolic engineering of the glycosylation machinery of a cell, including genetic manipulations of the oligosaccharide synthesis pathways to achieve altered glycosylation of glycoproteins expressed in these cells. Furthermore, glycoengineering includes the effects of mutations and cell environment on glycosylation. In particular, glycoengineering can result in altered glycosyltransferase activity in a cell, such as altered glucosaminyltransferase and/or fucosyltransferase activity.

The relative amount of non-fucosylated and/or bispecific is the percentage of carbohydrate structures lacking fucose and/or having a bisecting GlcNAc residue, related to all glycostructures identified in an N-Glycosidase F treated protein sample by MALDI-TOF MS.

In a particular embodiment, antibodies are also characterized in that they have been glycoengineered to have increased effector function and/or increased Fc receptor binding affinity. The term “effector function”, as used herein, refers to those biological activities attributable to the Fc region (a native sequence Fc region or amino acid sequence variant Fc region) of an antibody.

In a particular embodiment, the increased effector function is one or more of the following: increased Fc-mediated cellular cytotoxicity (including increased antibody-dependent cellular cytotoxicity (ADCC)), increased antibody-dependent cellular phagocytosis (ADCP), increased cytokine secretion, increased immune-complex-mediated antigen uptake by antigen-presenting cells, increased binding to natural killer (NK) cells, increased binding to macrophages, increased binding to monocytes, increased binding to polymorphonuclear cells, increased direct signaling inducing apoptosis, increased crosslinking of target-bound antibodies, increased dendritic cell maturation, or increased T cell priming. In an embodiment, the increased Fc receptor binding affinity is increased binding to a Fc activating receptor, and in a particular embodiment increased binding to FcγRIIIa.

A particular IgG-class anti-EGFR antibody useful in the formulations according to the invention is characterized in that it comprises the heavy chain variable domain of SEQ ID NO:38 and the light chain variable domain of SEQ ID NO:39, is humanized, and comprises the human heavy chain constant region Ig gamma-1, as set forth in SEQ ID NO:40. This antibody is termed “hu-ICR62 IgG1 anti-EGFR mAb”. hu-ICR62 IgG1 anti-EGFR mAb may or may not be glycoengineered as described above, to have an increased proportion of non-fucosylated oligosaccharides in the Fc region as compared to non-glycoengineered antibodies.

In a particular embodiment, the antibodies useful in the formulations according to the invention are produced by recombinant means, e.g. by those described in WO 2006/082515 and WO 2008/017963. Such methods are widely known in the art and comprise protein expression in prokaryotic or eukaryotic cells with subsequent isolation of the antibody polypeptide and usually purification to a pharmaceutically acceptable purity. For the protein expression, nucleic acids encoding light and heavy chains or fragments thereof are inserted into suitable expression vectors by standard methods. Expression is performed in appropriate host cells, which include cultured cells, for example, cultured mammalian cells such as CHO cells, HEK 293 cells, HEK293-EBNA cells, BHK cells, NS0 cells, SP2/0 cells, YO myeloma cells, P3X63 mouse myeloma cells, PER cells, PER.C6 cells or hybridoma cells, E. coli cells, yeast cells, insect cells and plant cells, but also cells comprised within a transgenic animal, transgenic plant or cultured plant or animal tissue. In a particular embodiment, host cells are CHO cells. The host cells used to express the antibodies may have been manipulated to have altered levels glycosyltransferase activity, such as altered glucosaminyltransferase and/or fucosyltransferase activity, to produce antibodies with an altered glycosylation pattern. In a particular embodiment, the host cells have been manipulated to express increased levels of one or more polypeptides having β(1,4)-N-acetylglucosaminyltransferase III (GnTIII) activity, and optionally one or more polypeptides having mannosidase II (ManII) activity. The polypeptide having GnTIII activity may be a fusion polypeptide comprising the catalytic domain of GnTIII and the Golgi localization domain of a heterologous Golgi resident polypeptide, for example, the Golgi localization domain of mannosidase II. The glycoengineering methods that can be employed with the IgG-class anti-EGFR antibodies useful in the present invention are described in WO 2006/082515, WO 2008/017963, U.S. Pat. No. 6,602,684, EP 1071700, WO 1999/54342, U.S. Pat. Appl. Publ. No. 2004/0241817, EP 1587921, WO 2004/065540, Umaña et al., Nature Biotechnol 17, 176-180 (1999), and Ferrara et al., Biotechn Bioeng 93, 851-861 (2006), the entire contents of each of which are incorporated herein by reference in their entirety.

The antibody is recovered from the cells (supernatant or cells after lysis) by standard techniques, e.g. Protein A affinity chromatography, size exclusion chromatography, and others well known in the art, e.g. as described in WO 2006/082515 and WO 2008/017963.

For the formulation according to the present invention the antibody is used at a concentration of about 1 to about 200 mg/ml, about 1 to about 100 mg/ml, about 10 to about 75 mg/ml, or about 20 to about 50 mg/ml.

The term “buffering agent” as used herein denotes a pharmaceutically acceptable excipient, which stabilizes the pH of a pharmaceutical preparation. Suitable buffers are well known in the art and can be found in the literature. For example, citrate salts, acetate salts, histidine salts, succinate salts, malate salts, phosphate salts or lactate salts, and/or the respective free acids or bases thereof, as well as mixtures of the various salts and/or acids and bases thereof can be employed. In a particular embodiment, pharmaceutically acceptable buffers comprise but are not limited to histidine buffers, citrate buffers, succinate buffers, acetate buffers and phosphate buffers. In a particular embodiment, buffers are acetate buffers, for example, sodium acetate buffer. Other particular buffers are histidine buffers, i.e. buffers having histidine, generally L-histidine, as buffering agent. A particular buffer is L-histidine/HCl buffer, comprising L-histidine or mixtures of L-histidine and L-histidine hydrochloride and pH adjustment achieved with hydrochloric acid. Unless otherwise indicated, the term “L-histidine” when used herein to describe a buffering agent, refers to L-histidine/HCl buffer. L-histidine/HCl buffer can be prepared by dissolving suitable amounts of L-histidine and L-histidine hydrochloride in water, or by dissolving a suitable amount of L-histidine in water and adjusting the pH to the desired value by addition of hydrochloric acid. The abovementioned buffers are generally used at a concentration of about 1 mM to about 100 mM, about 10 mM to about 50 mM, about 15 to 30 mM or 20 mM. Regardless of the buffer used, the pH can be adjusted to a value in the range from about 4.0 to about 7.0, about 5.0 to about 6.0, or about 5.5, with an acid or a base known in the art, e.g. hydrochloric acid, acetic acid, phosphoric acid, sulfuric acid and citric acid, sodium hydroxide and potassium hydroxide.

The term “surfactant” as used herein denotes a pharmaceutically acceptable, surface-active agent. In a particular embodiment, a non-ionic surfactant is used. Examples of pharmaceutically acceptable surfactants include, but are not limited to, polyoxyethylen-sorbitan fatty acid esters (Tween), polyoxyethylene alkyl ethers (Brij), alkylphenylpolyoxyethylene ethers (Triton X), polyoxyethylene-polyoxypropylene copolymers (Poloxamer, Pluronic), and sodium dodecyl sulphate (SDS). In a particular embodiment, polyoxyethylene-sorbitan fatty acid esters are polysorbate 20 (polyoxyethylene sorbitan monolaureate, sold under the trademark Tween 20™) and polysorbate 80 (polyoxyethylene sorbitan monooleate, sold under the trademark Tween 80™). In a particular embodiment, polyethylene-polypropylene copolymers are those sold under the names Pluronic® F68 or Poloxamer 188™. In a particular embodiment, polyoxyethylene alkyl ethers are those sold under the trademark Brij™. In a particular embodiment, alkylphenylpolyoxyethylene ethers are sold under the tradename Triton X, for example, p-tert-octylphenoxy polyethoxyethanol (sold under the tradename Triton X-100™). When polysorbate 20 (Tween 20™) and polysorbate 80 (Tween 80™) are used, they are generally used at a concentration range of about 0.001 to about 1%, about 0.01 to about 0.1% or about 0.02% to about 0.05%. In the formulation of the invention, the concentration of the surfactant is described as a percentage, expressed in weight/volume (w/v).

The term “stabilizer” as used herein denotes a pharmaceutically acceptable excipient, which protects the active pharmaceutical ingredient and/or the formulation from chemical and/or physical degradation during manufacturing, storage and application. Stabilizers include but are not limited to saccharides, amino acids, polyols, e.g. mannitol, sorbitol, xylitol, dextran, glycerol, arabitol, propylene glycol, polyethylene glycol, cyclodextrines, e.g. hydroxypropyl-β-cyclodextrine, sulfobutylethyl-β-cyclodextrine, β-cyclodextrine, polyethylenglycols, e.g. PEG 3000, PEG 3350, PEG 4000, PEG 6000, albumines, e.g. human serum albumin (HSA), bovine serum albumin (BSA), salts, e.g. sodium chloride, magnesium chloride, calcium chloride, chelators, e.g. EDTA as hereafter defined. As mentioned hereinabove, stabilizers can be present in the formulation in an amount of about 1 to about 500 mM, in an amount of about 10 to about 300 mM or in an amount of about 120 mM to about 300 mM. More than one stabilizer, selected from the same or from different groups, can be present in the formulation.

The term “saccharide” as used herein includes monosaccharides and oligosaccharides. A monosaccharide is a monomeric carbohydrate which is not hydrolysable by acids, including simple sugars and their derivatives, e.g. aminosugars. Saccharides are usually in their D conformation. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a linear chain. The monomeric saccharide units within an oligosaccharide can be identical or different. Depending on the number of monomeric saccharide units the oligosaccharide is a di-, tri-, tetra- penta- and so forth saccharide. In contrast to polysaccharides the monosaccharides and oligosaccharides are water soluble. Examples of oligosaccharides include sucrose, trehalose, lactose, maltose and raffinose. In a particular embodiment, saccharides are sucrose and trehalose (i.e. α,α-D-trehalose), for example, sucrose. Trehalose is available as trehalose dihydrate. Saccharides can be present in the formulation in an amount of about 100 to about 500 mM, in an amount of about 200 to about 300 mM or in an amount of about 240 mM.

The term “amino acid” as used herein denotes a pharmaceutically acceptable organic molecule possessing an amino moiety located at α-position to a carboxylic group. Examples of amino acids include but are not limited to arginine, glycine, ornithine, lysine, histidine, glutamic acid, asparagic acid, isoleucine, leucine, alanine, phenylalanine, tyrosine, tryptophane, methionine, serine, proline. In a particular embodiment, the amino acid employed is in each case the L-form. Basic amino acids, such as arginine, histidine, or lysine, may be employed in the form of their inorganic salts (for example, in the form of the hydrochloric acid salts, i.e. as amino acid hydrochlorides). In a particular embodiment, amino acids are arginine hydrochloride and methionine. In a particular embodiment, methionine is used at a concentration of about 10 to about 25 mM or about 10 mM. In a particular embodiment arginine hydrochloride is used at a concentration of about 100 to about 200 mM or at a concentration of about 155 mM.

A subgroup within the stabilizers are lyoprotectants. The term “lyoprotectant” denotes pharmaceutically acceptable excipients, which protect the labile active ingredient (e.g. a protein) against destabilizing conditions during the lyophilisation process, subsequent storage and reconstitution. Lyoprotectants comprise but are not limited to the group consisting of saccharides, polyols (such as e.g. sugar alcohols) and amino acids. In a particular embodiment, lyoprotectants can be selected from the group consisting of saccharides such as sucrose, trehalose, lactose, glucose, mannose, maltose, galactose, fructose, sorbose, raffinose, neuraminic acid, amino sugars such as glucosamine, galactosamine, N-methylglucosamine (“Meglumine”), polyols such as mannitol and sorbitol, and amino acids such as arginine and glycine or mixtures thereof. Lyoprotectants are generally used in an amount of about 10 to 500 mM, in an amount of about 10 to about 300 mM or in an amount of about 100 to about 300 mM.

A subgroup within the stabilizers are antioxidants. The term “antioxidant” denotes pharmaceutically acceptable excipients, which prevent oxidation of the active pharmaceutical ingredient. Antioxidants comprise but are not limited to ascorbic acid, gluthathione, cysteine, methionine, citric acid, EDTA. Antioxidants can be used in an amount of about 0.01 to about 100 mM, in an amount of about 5 to about 50 mM or in an amount of about 5 to about 25 mM.

The formulations according to the invention may also comprise one or more tonicity agents. The term “tonicity agents” denotes pharmaceutically acceptable excipients used to modulate the tonicity of the formulation. The formulation can be hypotonic, isotonic or hypertonic. Isotonicity in general relates to the osmotic pressure of a solution, usually relative to that of human blood serum (around 250-350 mOsmol/kg). The formulation according to the invention can be hypotonic, isotonic or hypertonic. In a particular embodiment, the formulation is isotonic. An isotonic formulation is liquid or liquid reconstituted from a solid form, e.g. from a lyophilized form, and denotes a solution having the same tonicity as some other solution with which it is compared, such as physiologic salt solution and the blood serum. Suitable tonicity agents comprise but are not limited to sodium chloride, potassium chloride, glycerine and any component from the group of amino acids or sugars, in particular glucose. Tonicity agents are generally used in an amount of about 5 mM to about 500 mM.

Within the stabilizers and tonicity agents there is a group of compounds which can function in both ways, i.e. they can at the same time be a stabilizer and a tonicity agent. Examples thereof can be found in the group of sugars, amino acids, polyols, cyclodextrines, polyethyleneglycols and salts. An example for a sugar which can at the same time be a stabilizer and a tonicity agent is trehalose.

The formulations may also contain adjuvants such as preservatives, wetting agents, emulsifying agents and dispersing agents. Prevention of presence of microorganisms may be ensured both by sterilization procedures, and by the inclusion of various antibacterial and antifungal agents, e.g. paraben, chlorobutanol, phenol, sorbic acid, and the like. Preservatives are generally used in an amount of about 0.001 to about 2% (w/v). Preservatives comprise but are not limited to ethanol, benzyl alcohol, phenol, m-cresol, p-chlor-m-cresol, methyl or propyl parabens, benzalkonium chloride.

In a first aspect the present invention relates to a pharmaceutical formulation comprising:

1 to 200 mg/ml of an IgG-class anti-EGFR antibody;
1 to 100 mM of a buffering agent;
0.001 to 1% (w/v) of a surfactant;
1 to 500 mM of at least one stabilizer
at a pH in the range of from 4.0 to 7.0.

In a particular embodiment, the concentration of the IgG-class anti-EGFR antibody comprised in the formulation according to the invention is in the range of 1 to 100 mg/ml, 10 to 75 mg/ml or 20 to 50 mg/ml. In a particular embodiment, the concentration of the IgG-class anti-EGFR antibody is 25 mg/ml.

In another particular embodiment, the buffering agent comprised in the formulation according to the invention is a histidine buffer, for example, a L-histidine/HCl buffer, or an acetate buffer or a sodium acetate buffer. In a particular embodiment, the buffering agent is L-histidine/HCl.

In a particular embodiment, the buffering agent is at a concentration of 10 to 50 mM, 15 to 30 mM or 20 mM.

In a particular embodiment, the buffering agent provides a pH of 5.0 to 6.0 or 5.5±0.3.

In a particular embodiment, the surfactant comprised in the formulation according to the invention is a polysorbate, for example, polysorbate 20 or polysorbate 80, or polysorbate 80.

In a particular embodiment, the surfactant is at a concentration of 0.01 to 0.1%, 0.02 to 0.05% (w/v) or 0.02 to 0.03%.

In yet another particular embodiment, the at least one stabilizer comprised in the formulation according to the invention is selected from the group of salts, for example, sodium chloride, saccharides, trehalose dihydrate or sucrose, and amino acids, such as arginine hydrochloride.

In a particular embodiment, the at least one stabilizer is at a concentration of 120 to 300 mM.

In a particular embodiment, the formulation according to the invention comprises a first stabilizer selected from the group of salts, saccharides and amino acids, and methionine as a second stabilizer.

In a particular embodiment, the first stabilizer is at a concentration of 120 to 300 mM, and the second stabilizer methionine is present at a concentration of 5 to 25 mM.

In a particular embodiment, the formulation according to the invention comprises a saccharide, for example, trehalose dihydrate or sucrose. In a particular embodiment, sucrose is used as a first stabilizer, and methionine as a second stabilizer. In a particular embodiment, the saccharide is at a concentration of about 240 mM, and methionine is at a concentration of about 10 mM.

In another particular embodiment, the IgG-class anti-EGFR antibody comprised in the formulation according to the invention is a humanized antibody and comprises

a) in the heavy chain variable domain a CDR1 of SEQ ID NO:1, a CDR2 of SEQ ID NO:16 and a CDR3 of SEQ ID NO:31, and
b) in the light chain variable domain a CDR1 of SEQ ID NO:33, a CDR2 of SEQ ID NO:34 and a CDR3 of SEQ ID NO:35.

In a particular embodiment, the IgG-class anti-EGFR antibody comprised in the formulation according to the invention is hu-ICR62 IgG1 anti-EGFR mAb.

In certain embodiments, the IgG-class anti-EGFR antibody comprised in the formulation according to the invention has been glycoengineered to have an increased proportion, for example, at least 20%, at least 50% or at least 70%, of non-fucosylated oligosaccharides it its Fc region, as compared to the non-glycoengineered antibody.

In a particular embodiment, the formulation according to the invention comprises:

10 to 50 mg/ml of an IgG-class anti-EGFR antibody;
15 to 30 mM of a buffering agent, selected from L-histidine and sodium acetate;
0.02 to 0.05% (w/v) of a surfactant, selected from polysorbate 20 and polysorbate 80;
120 to 300 mM of at least one stabilizer, selected from trehalose dihydrate, sucrose, arginine hydrochloride and sodium chloride;
optionally, 5 to 25 mM of methionine as a second stabilizer;
at a pH of 5.5±0.3.

In a particular embodiment, the formulation according to the invention comprises:

10 to 50 mg/ml of hu-ICR62 IgG1 anti-EGFR mAb;

### 15 to 30 mM L-histidine;

0.02 to 0.05% (w/v) polysorbate 80;
120 to 300 mM of at least one stabilizer, selected from trehalose dihydrate, sucrose, and arginine hydrochloride;
optionally, 5 to 25 mM of methionine as a second stabilizer;
at a pH of 5.5±0.3.

In yet another particular embodiment, the formulation according to the invention comprises:

10 to 50 mg/ml IgG-class anti-EGFR antibody, for example, hu-ICR62 IgG1 anti-EGFR mAb, 20 mM L-histidine, 240 mM trehalose dihydrate, 0.02 to 0.03% (w/v) polysorbate 80, at pH 5.5; or
10 to 50 mg/ml IgG-class anti-EGFR antibody, for example, hu-ICR62 IgG1 anti-EGFR mAb, 20 mM L-histidine, 155 mM arginine hydrochloride, 0.02% (w/v) polysorbate 80, at pH 5.5; or
10 to 50 mg/ml IgG-class anti-EGFR antibody, for example, hu-ICR62 IgG1 anti-EGFR mAb, 20 mM L-histidine, 240 mM trehalose dihydrate, 0.02 to 0.03% (w/v) polysorbate 80, 10 mM methionine at pH 5.5; or
10 to 50 mg/ml IgG-class anti-EGFR antibody, for example, hu-ICR62 IgG1 anti-EGFR mAb, 20 mM L-histidine, 240 mM sucrose, 0.02 to 0.03% (w/v) polysorbate 80, at pH 5.5; or
10 to 50 mg/ml IgG-class anti-EGFR antibody, for example, hu-ICR62 IgG1 anti-EGFR mAb, 20 mM L-histidine, 240 mM sucrose, 0.02 to 0.03% (w/v) polysorbate 80, 10 mM methionine at pH 5.5.

In a particular embodiment, the formulation according to the invention comprises:

20 to 50 mg/ml IgG-class anti-EGFR antibody, for example, hu-ICR62 IgG1 anti-EGFR mAb; 20 mM L-histidine;
0.02 to 0.03% (w/v) polysorbate 80; 240 mM of a first stabilizer, wherein said first stabilizer is a saccharide selected from trehalose dihydrate and sucrose;
10 mM of methionine as a second stabilizer; at a pH of 5.5±0.3.

In a particular embodiment, the first stabilizer is sucrose.

In certain embodiments, the formulation according to the invention does not comprise sodium chloride. In certain embodiments, the formulation does not comprise a divalent cation. In certain embodiments, the formulation does not comprise lactobionic acid. In certain embodiments, the formulation does not comprise a polyol. In certain embodiments, the formulation does not comprise a dextran.

The formulation according to the invention can be in a liquid form, in a lyophilized form or in a liquid form reconstituted from a lyophilized form. In certain embodiments, the formulation is in a liquid form.

The term “liquid” as used herein in connection with the formulation according to the invention denotes a formulation which is liquid at a temperature of at least about 2 to about 8° C. under atmospheric pressure.

The term “lyophilized” as used herein in connection with the formulation according to the invention denotes a formulation which is manufactured by freeze-drying methods known in the art per se. The solvent (e.g. water) is removed by freezing followed by sublimation of the ice under vacuum and desorption of residual water at elevated temperature. The lyophilizate usually has a residual moisture of about 0.1 to 5% (w/w) and is present as a powder or a physically stable cake. The lyophilizate is characterized by a fast dissolution after addition of a reconstitution medium.

The term “reconstituted form” as used herein in connection with the formulation according to the invention denotes a formulation which is lyophilized and re-dissolved by addition of reconstitution medium. Suitable reconstitution media comprise but are not limited to water for injection (WFI), bacteriostatic water for injection (BWFI), sodium chloride solutions (e.g. 0.9% (w/v) NaCl), glucose solutions (e.g. 5% glucose), surfactant-containing solutions (e.g. 0.01% polysorbate 20), pH-buffered solutions (eg. phosphate-buffered solutions).

The formulation according to the invention is physiologically well tolerated, can be prepared easily, can be dispensed precisely and is stable with respect to decomposition products and aggregates over the duration of storage, during repeated freezing and thawing cycles and mechanical stress. It is stable at refrigerator temperatures (2-8° C.) over a period of more than 1 year.

The invention further comprises a process for the preparation of the formulations according to the invention. Said process comprises buffer-exchanging the IgG-class anti-EGFR antibody against a diafiltration buffer containing the anticipated buffer composition, and, where required, concentration of the antibody by diafiltration, followed by adding the excipients (e.g. trehalose dihydrate, sucrose, arginine, sodium chloride, methionine) as stock solutions to the antibody solution, followed by adding the surfactant as stock solution to the antibody/excipient solution, and finally adjusting the antibody concentration to the desired final concentration using buffer solution, whereby also the final excipient and surfactant concentrations are reached.

Alternatively, the excipients can also be added as solids to the starting solution comprising the IgG-class anti-EGFR antibody. If the IgG-class anti-EGFR antibody is in the form of a solid, e.g. a lyophilizate, the formulation according to the invention can be prepared by firstly dissolving the antibody in water or buffer solution, optionally comprising one or more of the excipients, and subsequently adding the further excipients as stock solutions or solids. The IgG-class anti-EGFR antibody can advantageously also be dissolved directly in a solution comprising all further excipients. One or more of the excipients present in the formulation according to the invention may already be added during or at the end of the process for the preparation of the IgG-class anti-EGFR antibody, e.g. by dissolving the IgG-class anti-EGFR antibody directly in a solution comprising one, more than one or all of the excipients of the formulation in the final step of the purification carried out after the preparation of the antibody. If the solution comprising the antibody and the excipients does not yet have the desired pH, this is adjusted by addition of an acid or base, for example, using the acid or base already present in the buffer system. This is followed by sterile filtration.

The invention further comprises the use of the formulations according to the invention for the preparation of a medicament useful for treating diseases, particularly cell proliferation disorders, wherein EGFR is expressed, particularly wherein EGFR is abnormally expressed (e.g., overexpressed) compared to normal tissue of the same cell type. Such disorders include different types of cancer, such as cancers of the bladder, brain, head and neck, pancreas, lung, breast, ovary, colon, prostate, skin, and kidney. EGFR expression levels may be determined by methods known in the art and those described in WO 2006/082515 and WO 2008/017963 (e.g., via immunohistochemistry assay, immunofluorescence assay, immunoenzyme assay, ELISA, flow cytometry, radioimmunoassay, Western blot, ligand binding, kinase activity, etc.).

A composition of the present invention can be administered by a variety of methods known in the art. As will be appreciated by the skilled artisan, the route and/or mode of administration will vary depending upon the desired results.

To administer a composition of the invention by certain routes of administration, it may be necessary to dilute the composition in a diluent. Pharmaceutically acceptable diluents include saline, glucose, Ringer and aqueous buffer solutions.

In a particular embodiment, the formulation according to the invention is administered by intravenous (i.v.), subcutaneous (s.c.) or any other parental administration means such as those known in the pharmaceutical art.

The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The composition must be sterile and fluid to the extent that the composition is deliverable by syringe or an infusion system. In addition to water, the carrier can be an isotonic buffered saline solution, ethanol, polyol (e.g., glycerol, propylene glycol, and liquid polyethylene glycol, and the like), and suitable mixtures thereof.

The formulation according to the invention can be prepared by methods known in the art, e.g. ultrafiltration-diafiltration, dialysis, addition and mixing, lyophilisation, reconstitution, and combinations thereof. Examples of preparations of formulations according to the invention can be found hereinafter.

The examples explain the invention in more detail but should not be construed as limiting the scope of the invention. The disclosures of all patent and scientific literature cited herein are expressly incorporated in their entirety by reference.

### EXAMPLES

The IgG-class anti-EGFR antibody formulations according to the invention were developed based on the experimental results as provided below using the general preparatory and analytical methods and assays as outlined below.

### Example 1

### Preparation of the Components for the Formulation

The IgG-class anti-EGFR antibody hu-ICR62 IgG1 anti-EGFR mAb was manufactured by techniques generally known from the production of recombinant proteins. Techniques to manufacture this antibody are described in WO 2006/082515 and WO 2008/017963. Briefly, a genetically engineered Chinese hamster ovary cell line (CHO) prepared as described in WO 2006/082515 and WO 2008/017963 was expanded in cell culture from a master cell bank. The hu-ICR62 IgG1 anti-EGFR mAb was purified from the conditioned cell culture medium using protein A affinity chromatography on a MabSelect SuRe™ column (GE), followed by cation exchange chromatography on a Capto S™ column (GE) and a final anion exchange chromatographic step on a Capto Q™ column (GE). Viruses were removed by nanofiltration using a Viresolve® Pro membrane (Millipore) and the antibody was concentrated and transferred into the desired buffer by diafiltration. For preparing the formulations in accordance with these examples the hu-ICR62 IgG1 anti-EGFR mAb antibody was provided at a concentration of approx. 20 mg/ml in a 20 mM histidine buffer (a L-histidine/HCl buffer) at a pH of approximately 6.0.

The excipients of the formulation in accordance with the present invention are widely used in the practice and known to the person skilled in the art. There is therefore no need to explain them here in detail.

Liquid drug product formulations according to the invention were developed as follows.

### Example 2

### Preparation of the Liquid Formulations

For the preparation of the liquid formulations hu-ICR62 IgG1 anti-EGFR mAb was buffer-exchanged against a diafiltration buffer containing the anticipated buffer composition and where required, concentrated by diafiltration to an antibody concentration of approx. 50-80 mg/ml. After completion of the diafiltration operation, the excipients (e.g. trehalose, sodium chloride, methionine) were added as stock solutions to the antibody solution. The surfactant was then added as a 50 to 200-fold stock solution. Finally, the protein concentration was adjusted with a buffer to the final hu-ICR62 IgG1 anti-EGFR mAb antibody concentration of approx. 25 mg/ml or approx. 50 mg/ml.

All formulations were sterile-filtered through 0.22 μm low protein binding filters and aseptically filled into sterile 6 ml glass vials closed with ETFE (copolymer of ethylene and tetrafluoroethylene)-coated rubber stoppers and aluminium crimp caps. The fill volume was approx. 2.4 ml. These formulations were stored at different ICH climate conditions (5° C., 25° C. and 40° C.) for different intervals of time and stressed by shaking (1 week at a shaking frequency of 200 min⁻¹ at 5° C. and 25° C.) and freeze-thaw stress methods. The samples were analyzed before and after applying the stress tests by the following analytical methods:

1) UV spectrophotometry;

### 2) Size Exclusion Chromatography (SEC);

### 3) Ion Exchange Chromatography (IEC);

4) measurement of the turbidity of the solution;
5) inspection for visible particles.

UV spectroscopy, used for determination of protein content, was performed on a Perkin Elmer λ35 UV spectrophotometer in a wavelength range from 240 nm to 400 nm. Neat protein samples were diluted to approx. 0.5 mg/ml with the corresponding formulation buffer. The protein concentration was calculated according to Equation 1.

$$Protein content = \frac{( {A}_{280 nm} - {A}_{320 nm} ) \times dil . factor}{ɛ [ \frac{{cm}^{2}}{mg} ] \times {d}_{cm}} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient E.

Size Exclusion Chromatography (SEC) was used to detect soluble high molecular weight species (aggregates) and low molecular weight hydrolysis products (LMW) in the formulations. The method was performed on a Waters Alliance 2695 HPLC instrument with a Waters W2487 Dual Absorbance Detector and equipped with a TosoHaas TSK Gel G3000SWXL column. Intact monomer, aggregates and hydrolysis products were separated by an isocratic elution profile, using 200 mM sodium phosphate, pH 7.0 as mobile phase, and were detected at a wavelength of 280 nm.

Ion Exchange Chromatography (IEC) was performed to detect chemical degradation products altering the net charge of hu-ICR62 IgG1 anti-EGFR mAb in the formulations. The method used a suitable HPLC instrument equipped with a UV detector (detection wavelength 280 nm) and a Dionex ProPac WCX-10 column (4 mm×250 mm). 10 mM sodium phosphate buffer pH 6.0 in water and 10 mM sodium phosphate buffer pH 6.0+750 mM NaCl were used as mobile phases A and B, respectively, with a flow rate of 1.0 mL/min.

For the determination of the turbidity, opalescence was measured in FTU (turbidity units) using a HACH 2100AN turbidimeter at room temperature.

Samples were analyzed for visible particles by using a Seidenader V90-T visual inspection instrument.

The results of the stability testing for the Formulations A to L are provided in Tablel added below.

The results show, that for obtaining maximum antibody stability and antibody formulations free from particles, the use of L-histidine/HCl buffer is more favorable than the use of sodium acetate buffer, while saccharides such as trehalose dihydrate and sucrose are the most favorable stabilizers, and polysorbate 80 is the most favorable surfactant.

TABLE 1
Composition and stability data of liquid anti-EGFR antibody drug product formulations according to this invention

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Acidic | Main | Basic | Turbidity | Visible |
| condition | Time | (mg/ml) | (%) | (%) | (%) | Peak (%) | Peak (%) | Peak (%) | (FTU) | particles |

Formulation A is a liquid formulation with the composition 25 mg/ml
hu-ICR62 IgG1 anti-EGFR mAb, 20 mM sodium acetate pH 5.3, 140 mM
sodium chloride, 0.02% polysorbate 20.

| | | | | | | | | | | |
|---|---|---|---|---|---|---|---|---|---|---|
| — | Initial | 25.6 | 1.4 | 98.6 | 0 | 35.6 | 60.2 | 4.3 | 8.9 | Free from particles |
| Shaking 5° C. | 1 week | 25.7 | 1.5 | 98.4 | 0.1 | 36.3 | 59.5 | 4.2 | 8.9 | Free from particles |
| Shaking 25° C. | 1 week | 25.6 | 1.7 | 98.2 | 0.1 | 36.7 | 58.1 | 5.2 | 8.8 | Free from particles |
| Freezing/ | (5 cycles) | 25.6 | 1.8 | 98.1 | 0.1 | 36.6 | 59.1 | 4.4 | 9.6 | Free from particles |
| Thawing | | | | | | | | | | |
| 2-8° C. | 9 weeks | 25.5 | 1.7 | 98.2 | 0.1 | 36.2 | 59.1 | 4.7 | 8.9 | Free from particles |
| | 32 weeks | 25.3 | 2.0 | 97.9 | 0.1 | 32.1 | 64.5 | 3.4 | 9.8 | With many particles |
| | 51 weeks | 25.4 | 2.2 | 97.7 | 0.1 | 35.6 | 59.1 | 5.3 | 10.0 | With many particles |
| | 74 weeks | nd | 2.3 | 97.6 | 0.1 | 37.2 | 57.8 | 5.1 | 10.2 | With many particles |
| 25° C. | 9 weeks | nd | 2.2 | 97.5 | 0.3 | 41.4 | 53.3 | 5.3 | 10.0 | Free from particles |
| | 32 weeks | nd | 2.6 | 95.0 | 2.4 | 52.0 | 43.3 | 4.7 | 9.8 | Free from particles |
| | 51 weeks | nd | 2.9 | 93.0 | 4.1 | 59.5 | 35.2 | 5.3 | 9.7 | Free from particles |
| | 74 weeks | nd | 3.1 | 91.8 | 5.1 | 66.1 | 28.7 | 5.2 | 9.9 | With many particles |
| 40° C. | 9 weeks | 25.4 | 2.9 | 95.7 | 1.4 | nd | nd | nd | 9.4 | Free from particles |
| | 32 weeks | nd | 5.3 | 82.1 | 12.6 | nd | nd | nd | 10.7 | Free from particles |
| | 51 weeks | nd | 8.0 | 74.4 | 17.6 | nd | nd | nd | 11.8 | Free from particles |
| | 74 weeks | nd | 12.1 | 66.8 | 21.1 | nd | nd | nd | 13.8 | With many particles |

Formulation B is a liquid formulation with the composition 25 mg/ml
hu-ICR62 IgG1 anti-EGFR mAb, 20 mM sodium acetate pH 5.3, 240 mM
trehalose dihydrate, 0.02% polysorbate 20.

| | | | | | | | | | | |
|---|---|---|---|---|---|---|---|---|---|---|
| — | Initial | 25.6 | 1.2 | 98.8 | 0 | 36.1 | 60.0 | 3.9 | 2.7 | Free from particles |
| Shaking 5° C. | 1 week | 25.7 | 1.2 | 98.7 | 0.1 | 36.6 | 59.7 | 3.7 | 2.9 | Free from particles |
| Shaking 25° C. | 1 week | 25.8 | 1.3 | 98.6 | 0.1 | 37.0 | 58.7 | 4.2 | 2.7 | Practically free from |
| | | | | | | | | | | particles |
| Freezing/ | (5 cycles) | 26.2 | 1.2 | 98.8 | 0 | 37.1 | 58.9 | 4.0 | 2.7 | Practically free from |
| Thawing | | | | | | | | | | particles |
| 2-8° C. | 9 weeks | 25.8 | 1.3 | 98.6 | 0.1 | 36.9 | 59.0 | 4.1 | 3.1 | Free from particles |
| | 32 weeks | 25.6 | 1.4 | 98.5 | 0.1 | 34.1 | 62.8 | 3.1 | 3.1 | With many particles |
| | 51 weeks | 25.4 | 1.5 | 98.4 | 0.1 | 37.0 | 58.5 | 4.5 | 3.5 | With many particles |
| | 74 weeks | nd | 1.5 | 98.4 | 0.1 | 39.0 | 56.9 | 4.2 | 4.3 | With many particles |
| 25° C. | 9 weeks | nd | 1.4 | 98.4 | 0.2 | 43.0 | 52.7 | 4.3 | 2.8 | Free from particles |
| | 32 weeks | nd | 1.6 | 95.8 | 2.6 | 55.9 | 41.3 | 2.8 | 3.5 | Free from particles |
| | 51 weeks | nd | 1.7 | 94.6 | 3.7 | 64.0 | 32.3 | 3.7 | 3.5 | Practically free from |
| | | | | | | | | | | particles |
| | 74 weeks | nd | 1.8 | 93.7 | 4.5 | 71.0 | 25.5 | 3.6 | 3.6 | With many particles |
| 40° C. | 9 weeks | 25.7 | 1.5 | 97.4 | 1.1 | nd | nd | nd | 3.0 | Practically free from |
| | | | | | | | | | | particles |
| | 32 weeks | nd | 2.5 | 87.0 | 10.5 | nd | nd | nd | 3.7 | Free from particles |
| | 51 weeks | nd | 4.1 | 80.6 | 15.3 | nd | nd | nd | 3.7 | Practically free from |
| | | | | | | | | | | particles |
| | 74 weeks | nd | 6.3 | 74.6 | 19.1 | nd | nd | nd | 3.9 | With many particles |

Formulation C is a liquid formulation with the composition 25 mg/ml
hu-ICR62 IgG1 anti-EGFR mAb, 20 mM sodium acetate pH 5.3, 155 mM
arginine hydrochloride, 0.02% polysorbate 20.

| | | | | | | | | | | |
|---|---|---|---|---|---|---|---|---|---|---|
| — | Initial | 25.7 | 1.2 | 98.8 | 0 | 35.8 | 60.4 | 3.7 | 7.0 | Free from particles |
| Shaking 5° C. | 1 week | 25.9 | 1.2 | 98.7 | 0.1 | 36.2 | 59.7 | 4.1 | 7.4 | Free from particles |
| Shaking 25° C. | 1 week | 25.9 | 1.3 | 98.6 | 0.1 | 36.2 | 58.9 | 4.9 | 7.3 | Practically free from |
| | | | | | | | | | | particles |
| Freezing/ | (5 cycles) | 25.9 | 1.2 | 98.7 | 0.1 | 36.7 | 59.1 | 4.2 | 7.7 | Practically free from |
| Thawing | | | | | | | | | | particles |
| 2-8° C. | 9 weeks | 25.3 | 1.3 | 98.6 | 0.1 | 35.9 | 59.7 | 4.4 | 7.5 | Free from particles |
| | 32 weeks | 25.6 | 1.5 | 98.4 | 0.1 | 33.1 | 62.9 | 4.0 | 8.2 | With many particles |
| | 51 weeks | 25.5 | 1.6 | 98.3 | 0.1 | 35.0 | 59.8 | 5.2 | 8.8 | With many particles |
| | 74 weeks | nd | 1.7 | 98.2 | 0.1 | 36.7 | 58.3 | 5.0 | 8.1 | With many particles |
| 25° C. | 9 weeks | nd | 1.5 | 98.2 | 0.3 | 41.0 | 53.9 | 5.1 | 8.0 | Free from particles |
| | 32 weeks | nd | 1.7 | 95.7 | 2.6 | 52.0 | 43.7 | 4.3 | 10.0 | Free from particles |
| | 51 weeks | nd | 1.8 | 93.9 | 4.3 | 59.3 | 35.5 | 5.2 | 8.0 | Free from particles |
| | 74 weeks | nd | 1.9 | 93.0 | 5.1 | 66.3 | 28.7 | 5.0 | 8.2 | Free from particles |
| 40° C. | 9 weeks | 25.2 | 1.9 | 96.6 | 1.5 | nd | nd | nd | 8.1 | Free from particles |
| | 32 weeks | nd | 4.0 | 82.9 | 13.1 | nd | nd | nd | 8.6 | Free from particles |
| | 51 weeks | nd | 7.3 | 73.6 | 19.1 | nd | nd | nd | 10.1 | Free from particles |
| | 74 weeks | nd | 11.7 | 66.0 | 22.3 | nd | nd | nd | 13.4 | With many particles |

Formulation D is a liquid formulation with the composition 25 mg/ml
hu-ICR62 IgG1 anti-EGFR mAb, 20 mM L-histidine pH 5.5, 140 mM sodium
chloride, 0.02% polysorbate 80.

| | | | | | | | | | | |
|---|---|---|---|---|---|---|---|---|---|---|
| — | Initial | 25.5 | 1.3 | 98.7 | 0 | 36.3 | 59.8 | 3.9 | 8.6 | Free from particles |
| Shaking 5° C. | 1 week | 25.4 | 1.4 | 98.5 | 0.1 | 36.6 | 59.5 | 4.0 | 8.8 | Free from particles |
| Shaking 25° C. | 1 week | 25.4 | 1.5 | 98.4 | 0.1 | 36.9 | 58.4 | 4.8 | 9.2 | Practically free from |
| | | | | | | | | | | particles |
| Freezing/ | (5 cycles) | 25.4 | 1.5 | 98.4 | 0.1 | 36.8 | 59.1 | 4.2 | 8.9 | Free from particles |
| Thawing | | | | | | | | | | |
| 2-8° C. | 9 weeks | 25.0 | 1.5 | 98.4 | 0.1 | 36.9 | 58.9 | 4.2 | 9.0 | Free from particles |
| | 32 weeks | 25.3 | 1.8 | 98.1 | 0.1 | 34.7 | 61.9 | 3.4 | 9.6 | Practically free from |
| | | | | | | | | | | particles |
| | 51 weeks | 25.4 | 1.9 | 98.0 | 0.1 | 37.4 | 58.0 | 4.5 | 10.1 | Free from particles |
| | 74 weeks | nd | 1.9 | 98.0 | 0.1 | 39.7 | 56.0 | 4.3 | 10.0 | Free from particles |
| 25° C. | 9 weeks | nd | 1.8 | 98.0 | 0.2 | 43.3 | 52.1 | 4.6 | 9.0 | Free from particles |
| | 32 weeks | nd | 2.2 | 94.9 | 2.9 | 58.1 | 38.1 | 3.6 | 9.7 | Free from particles |
| | 51 weeks | nd | 2.4 | 93.2 | 4.4 | 65.5 | 30.0 | 4.5 | 9.3 | With many particles |
| | 74 weeks | nd | 2.5 | 92.4 | 5.1 | 73.5 | 22.6 | 3.9 | 10.7 | Practically free from |
| | | | | | | | | | | particles |
| 40° C. | 9 weeks | 25.24 | 2.7 | 95.7 | 1.6 | nd | nd | nd | 9.6 | Free from particles |
| | 32 weeks | nd | 5.8 | 81.3 | 12.9 | nd | nd | nd | 11.8 | Free from particles |
| | 51 weeks | nd | 9.1 | 72.2 | 18.7 | nd | nd | nd | 13.1 | Free from particles |
| | 74 weeks | nd | 13.5 | 64.6 | 21.9 | nd | nd | nd | 16.7 | With many particles |

Formulation E is a liquid formulation with the composition 25 mg/ml
hu-ICR62 IgG1 anti-EGFR mAb, 20 mM L-histidine pH 5.5, 240 mM
trehalose dihydrate, 0.02% polysorbate 80.

| | | | | | | | | | | |
|---|---|---|---|---|---|---|---|---|---|---|
| — | Initial | 25.7 | 1.1 | 98.9 | 0 | 36.4 | 60.0 | 4.0 | 2.9 | Free from particles |
| Shaking 5° C. | 1 week | 25.6 | 1.2 | 98.7 | 0.1 | 36.9 | 59.2 | 3.9 | 2.9 | Free from particles |
| Shaking 25° C. | 1 week | 25.6 | 1.2 | 98.7 | 0.1 | 37.3 | 58.7 | 4.1 | 3.0 | Free from particles |
| Freezing/ | (5 cycles) | 25.7 | 1.2 | 98.8 | 0 | 37.1 | 59.2 | 3.7 | 3.0 | Practically free from |
| Thawing | | | | | | | | | | particles |
| 2-8° C. | 9 weeks | 25.6 | 1.3 | 98.6 | 0.1 | 37.4 | 58.8 | 3.9 | 3.0 | Free from particles |
| | 32 weeks | 25.6 | 1.4 | 98.5 | 0.1 | 35.4 | 61.6 | 3.1 | 3.4 | Free from particles |
| | 51 weeks | 25.6 | 1.4 | 98.5 | 0.1 | 38.4 | 57.6 | 4.0 | 3.3 | Free from particles |
| | 74 weeks | nd | 1.4 | 98.5 | 0.1 | 40.1 | 56.0 | 3.9 | 3.7 | Free from particles |
| 25° C. | 9 weeks | nd | 1.3 | 98.5 | 0.2 | 44.1 | 52.0 | 3.9 | 3.2 | Free from particles |
| | 32 weeks | nd | 1.4 | 95.7 | 2.9 | 59.5 | 37.7 | 2.8 | 3.9 | Free from particles |
| | 51 weeks | nd | 1.6 | 93.9 | 4.5 | 67.5 | 29.1 | 3.4 | 3.7 | Free from particles |
| | 74 weeks | nd | 1.5 | 93.2 | 5.3 | 74.9 | 22.2 | 2.9 | 3.8 | Free from particles |
| 40° C. | 9 weeks | 25.8 | 1.4 | 97.2 | 1.4 | nd | nd | nd | 3.3 | Free from particles |
| | 32 weeks | nd | 3.4 | 84.9 | 11.7 | nd | nd | nd | 4.3 | Practically free from |
| | | | | | | | | | | particles |
| | 51 weeks | nd | 6.2 | 76.6 | 17.2 | nd | nd | nd | 4.8 | Practically free from |
| | | | | | | | | | | particles |
| | 74 weeks | nd | 11.6 | 65.5 | 22.9 | nd | nd | nd | 5.4 | With many particles |

Formulation F is a liquid formulation with the composition 25 mg/ml
hu-ICR62 IgG1 anti-EGFR mAb, 20 mM L-histidine pH 5.5, 155 mM
arginine hydrochloride, 0.02% polysorbate 80.

| | | | | | | | | | | |
|---|---|---|---|---|---|---|---|---|---|---|
| — | Initial | 25.3 | 1.2 | 98.8 | 0 | 36.5 | 59.9 | 3.7 | 7.8 | Free from particles |
| Shaking 5° C. | 1 week | 25.6 | 1.2 | 98.7 | 0.1 | 36.6 | 59.4 | 4.0 | 7.6 | Free from particles |
| Shaking 25° C. | 1 week | 25.6 | 1.3 | 98.6 | 0.1 | 36.8 | 58.5 | 4.7 | 7.4 | Free from particles |
| Freezing/ | (5 cycles) | 25.6 | 1.2 | 98.7 | 0.1 | 37.2 | 58.8 | 4.0 | 7.8 | Practically free from |
| Thawing | | | | | | | | | | particles |
| 2-8° C. | 9 weeks | 25.0 | 1.4 | 98.5 | 0.1 | 36.8 | 59.2 | 3.7 | 7.4 | Free from particles |
| | 32 weeks | 25.3 | 1.5 | 98.4 | 0.1 | 35.3 | 61.5 | 3.2 | 8.4 | Free from particles |
| | 51 weeks | 25.4 | 1.6 | 98.3 | 0.1 | 37.2 | 58.3 | 4.5 | 8.1 | Free from particles |
| | 74 weeks | nd | 1.6 | 98.3 | 0.1 | 39.3 | 56.4 | 4.3 | 8.0 | Free from particles |
| 25° C. | 9 weeks | nd | 1.4 | 98.3 | 0.3 | 42.9 | 52.6 | 4.5 | 8.0 | Free from particles |
| | 32 weeks | nd | 1.6 | 95.4 | 3.0 | 57.9 | 38.4 | 3.7 | 8.1 | Free from particles |
| | 51 weeks | nd | 1.7 | 93.8 | 4.5 | 66.2 | 29.9 | 3.8 | 8.2 | With many particles |
| | 74 weeks | nd | 1.7 | 93.0 | 5.3 | 72.9 | 23.4 | 3.7 | 8.8 | Free from particles |
| 40° C. | 9 weeks | 25.2 | 2.0 | 96.2 | 1.8 | nd | nd | nd | 8.3 | Free from particles |
| | 32 weeks | nd | 4.3 | 82.6 | 13.1 | nd | nd | nd | 9.7 | Practically free from |
| | | | | | | | | | | particles |
| | 51 weeks | nd | 7.3 | 73.3 | 19.4 | nd | nd | nd | 10.8 | Practically free from |
| | | | | | | | | | | particles |
| | 74 weeks | nd | 11.6 | 65.5 | 22.9 | nd | nd | nd | 12.7 | With many particles |

Formulation G is a liquid formulation with the composition 50 mg/ml
hu-ICR62 IgG1 anti-EGFR mAb, 20 mM L-histidine pH 5.5, 240 mM
trehalose dihydrate, 0.03% polysorbate 80.

| | | | | | | | | | | |
|---|---|---|---|---|---|---|---|---|---|---|
| — | Initial | 58.7 | 1.6 | 98.3 | 0.1 | 34.7 | 56.1 | 9.3 | 4.1 | Practically free from |
| | | | | | | | | | | particles |
| Shaking 5° C. | 1 week | 58.6 | 1.6 | 98.3 | 0.1 | 34.8 | 56.0 | 9.2 | 4.7 | Free from particles |
| Shaking 25° C. | 1 week | 58.5 | 1.7 | 98.2 | 0.1 | 35.3 | 55.6 | 9.1 | 4.3 | Free from particles |
| Freezing/ | (5 cycles) | 58.4 | 1.6 | 98.3 | 0.1 | 34.9 | 56.0 | 9.1 | 4.0 | Free from particles |
| Thawing | | | | | | | | | | |
| 2-8° C. | 16 weeks | 58.3 | 1.8 | 98 | 0.2 | 35.6 | 55.1 | 9.3 | 4.0 | Free from particles |
| | 29 weeks | nd | 1.9 | 97.9 | 0.2 | 36.5 | 54.0 | 9.5 | 4.3 | Free from particles |
| 25° C. | 16 weeks | nd | 1.8 | 97.7 | 0.5 | 35.6 | 55.1 | 9.3 | 3.8 | Free from particles |
| | 29 weeks | nd | 1.9 | 97.4 | 0.7 | 52.5 | 40.3 | 7.3 | 4.3 | Free from particles |
| 40° C. | 16 weeks | nd | 3.3 | 86.7 | 10.0 | nd | nd | nd | 4.6 | Free from particles |

Formulation H is a liquid formulation with the composition 50 mg/ml
hu-ICR62 IgG1 anti-EGFR mAb, 20 mM L-histidine pH 5.5, 240 mM trehalose
dihydrate, 0.03% polysorbate 80, 10 mM methionine.

| | | | | | | | | | | |
|---|---|---|---|---|---|---|---|---|---|---|
| — | Initial | 58.4 | 1.6 | 98.3 | 0.1 | 34.6 | 56.2 | 9.2 | 3.8 | Free from particles |
| Shaking 5° C. | 1 week | 60.4 | 1.6 | 98.3 | 0.1 | 35.0 | 56.2 | 8.9 | 4.2 | Free from particles |
| Shaking 25° C. | 1 week | 60.0 | 1.5 | 98.4 | 0.1 | 35.3 | 55.8 | 8.9 | 4.2 | Free from particles |
| Freezing/ | (5 cycles) | 60.2 | 1.5 | 98.4 | 0.1 | 34.8 | 56.3 | 8.9 | 4.4 | Free from particles |
| Thawing | | | | | | | | | | |
| 2-8° C. | 16 weeks | 58.4 | 1.7 | 98.1 | 0.2 | 35.6 | 55.2 | 9.2 | 4.0 | Free from particles |
| | 29 weeks | nd | 1.7 | 98.1 | 0.2 | 36.2 | 54.5 | 9.3 | 3.9 | Free from particles |
| 25° C. | 16 weeks | nd | 1.7 | 97.8 | 0.5 | 44.6 | 47.6 | 7.9 | 4.1 | Practically free from |
| | | | | | | | | | | particles |
| | 29 weeks | nd | 1.8 | 97.5 | 0.7 | 51.6 | 41.3 | 7.1 | 4.2 | Free from particles |
| 40° C. | 16 weeks | nd | 2.6 | 88 | 9.4 | nd | nd | nd | 4.9 | Free from particles |

Formulation I is a liquid formulation with the composition 25 mg/ml
hu-ICR62 IgG1 anti-EGFR mAb, 20 mM L-histidine pH 5.5, 240 mM trehalose
dihydrate, 0.02% polysorbate 80, 10 mM methionine.

| | | | | | | | | | | |
|---|---|---|---|---|---|---|---|---|---|---|
| — | Initial | 24.1 | 1.3 | 98.5 | 0.1 | 49.0 | 47.9 | 3.0 | 3.6 | Free from particles |
| Shaking 5° C. | 1 week | 24.2 | 1.3 | 98.5 | 0.1 | 49.5 | 47.2 | 3.3 | 3.4 | Practically free from |
| | | | | | | | | | | particles |
| Shaking 25° C. | 1 week | 24.3 | 1.3 | 98.5 | 0.2 | 49.9 | 46.6 | 3.5 | 3.4 | Practically free from |
| | | | | | | | | | | particles |
| Freezing/ | (5 cycles) | 24.1 | 1.3 | 98.5 | 0.2 | 49.4 | 47.1 | 3.5 | 3.5 | Free from particles |
| Thawing | | | | | | | | | | |
| 2-8° C. | 4 weeks | nd | Nd | nd | nd | nd | nd | nd | nd | nd |
| 25° C. | 4 weeks | nd | 1.3 | 98.5 | 0.2 | 51.6 | 45.0 | 3.4 | 3.6 | Free from particles |
| 40° C. | 4 weeks | nd | 1.0 | 98.3 | 0.7 | 69.0 | 27.7 | 3.4 | 3.5 | Free from particles |
| 2-8° C. | 12 weeks | nd | 1.3 | 98.5 | 0.2 | 48.9 | 47.4 | 3.7 | 3.7 | Free from particles |
| 25° C. | 12 weeks | nd | 1.3 | 98.4 | 0.3 | 54.9 | 41.5 | 3.7 | 3.7 | Free from particles |
| 40° C. | 12 weeks | nd | 1.1 | 93.9 | 5.1 | 84.6 | 12.8 | 2.6 | 4.0 | Free from particles |
| 2-8° C. | 24 weeks | nd | 1.3 | 98.5 | 0.2 | 50.2 | 46.2 | 3.6 | 3.3 | Free from particles |
| 25° C. | 24 weeks | nd | 1.3 | 98.3 | 0.4 | 61.0 | 35.67 | 3.4 | 3.4 | Free from particles |
| 40° C. | 24 weeks | nd | 1.3 | 89.3 | 9.4 | nd | nd | nd | 3.5 | Free from particles |
| 2-8° C. | 36 weeks | nd | 1.3 | 98.5 | 0.2 | 50.2 | 46.5 | 3.4 | 3.2 | Practically free from |
| | | | | | | | | | | particles |
| 25° C. | 36 weeks | nd | 1.2 | 98.2 | 0.6 | 65.0 | 32.1 | 2.9 | 3.3 | Free from particles |
| 2-8° C. | 55 weeks | nd | 1.3 | 98.5 | 0.2 | 50.1 | 46.3 | 3.6 | 3.6 | Free from particles |
| 25° C. | 55 weeks | nd | 1.2 | 98.0 | 0.8 | 70.6 | 26.3 | 3.1 | 3.8 | Free from particles |

Formulation J is a liquid formulation with the composition 25 mg/ml
hu-ICR62 IgG1 anti-EGFR mAb, 20 mM L-histidine pH 5.5, 240 mM sucrose,
0.02% polysorbate 80, 10 mM methionine.

| | | | | | | | | | | |
|---|---|---|---|---|---|---|---|---|---|---|
| — | Initial | 24.3 | 1.3 | 98.5 | 0.1 | 49.2 | 47.8 | 3.1 | 3.5 | Free from particles |
| Shaking 5° C. | 1 week | 24.2 | 1.3 | 98.5 | 0.2 | 49.4 | 47.1 | 3.5 | 3.7 | Practically free from |
| | | | | | | | | | | particles |
| Shaking 25° C. | 1 week | 24.2 | 1.3 | 98.6 | 0.2 | 49.9 | 46.6 | 3.6 | 4.0 | Practically free from |
| | | | | | | | | | | particles |
| Freezing/ | (5 cycles) | 24.1 | 1.3 | 98.5 | 0.2 | 49.3 | 48.6 | 2.1 | 3.6 | Practically free from |
| Thawing | | | | | | | | | | particles |
| 2-8° C. | 4 weeks | nd | nd | nd | nd | nd | nd | nd | nd | nd |
| 25° C. | 4 weeks | nd | 1.3 | 98.5 | 0.2 | 51.3 | 45.3 | 3.3 | 3.4 | Free from particles |
| 40° C. | 4 weeks | nd | 1.0 | 98.3 | 0.7 | 69.2 | 27.4 | 3.4 | 3.6 | Free from particles |
| 2-8° C. | 12 weeks | nd | 1.3 | 98.5 | 0.2 | 49.1 | 47.0 | 3.9 | 3.8 | Practically free from |
| | | | | | | | | | | particles |
| 25° C. | 12 weeks | nd | 1.2 | 98.5 | 0.3 | 54.7 | 41.6 | 3.7 | 4.0 | Practically free from |
| | | | | | | | | | | particles |
| 40° C. | 12 weeks | nd | 1.1 | 93.6 | 5.4 | 85.2 | 12.1 | 2.7 | 3.8 | Free from particles |
| 2-8° C. | 24 weeks | nd | 1.3 | 98.5 | 0.2 | 50.2 | 46.2 | 3.6 | 3.4 | Free from particles |
| 25° C. | 24 weeks | nd | 1.2 | 98.3 | 0.4 | 60.8 | 35.8 | 3.4 | 3.4 | Free from particles |
| 40° C. | 24 weeks | nd | 1.5 | 89.1 | 9.4 | nd | nd | nd | 3.9 | Free from particles |
| 2-8° C. | 36 weeks | nd | 1.3 | 98.5 | 0.2 | 50.2 | 46.2 | 3.6 | 3.1 | Free from particles |
| 25° C. | 36 weeks | nd | 1.2 | 98.2 | 0.6 | 64.8 | 32.2 | 3.0 | 3.5 | Free from particles |
| 2-8° C. | 55 weeks | nd | 1.3 | 98.5 | 0.2 | 50.2 | 46.3 | 3.5 | 3.7 | Free from particles |
| 25° C. | 55 weeks | nd | 1.2 | 98.0 | 0.8 | 71.2 | 26.2 | 2.7 | 4.0 | Free from particles |

Formulation K is a liquid formulation with the composition 25 mg/ml
hu-ICR62 IgG1 anti-EGFR mAb, 20 mM L-histidine pH 5.5, 240 mM
trehalose dihydrate, 0.02% polysorbate 80.

| | | | | | | | | | | |
|---|---|---|---|---|---|---|---|---|---|---|
| — | Initial | 24.2 | 1.3 | 98.5 | 0.1 | 49.4 | 47.5 | 3.2 | 3.6 | Free from particles |
| Shaking 5° C. | 1 week | 24.2 | 1.3 | 98.5 | 0.2 | 49.4 | 47.1 | 3.5 | 3.7 | Practically free from |
| | | | | | | | | | | particles |
| Shaking 25° C. | 1 week | 24.2 | 1.3 | 98.5 | 0.2 | 50.0 | 46.5 | 3.5 | 3.7 | Free from particles |
| Freezing/ | (5 cycles) | 23.9 | 1.3 | 98.5 | 0.2 | 49.6 | 47.0 | 3.4 | 3.5 | Practically free from |
| Thawing | | | | | | | | | | particles |
| 2-8° C. | 4 weeks | nd | nd | nd | nd | nd | nd | nd | nd | nd |
| 25° C. | 4 weeks | nd | 1.3 | 98.5 | 0.2 | 51.8 | 44.8 | 3.4 | 3.6 | Free from particles |
| 40° C. | 4 weeks | nd | 1.1 | 98.2 | 0.7 | 69.1 | 27.4 | 3.5 | 3.6 | Free from particles |
| 2-8° C. | 12 weeks | nd | 1.3 | 98.5 | 0.2 | 49.3 | 47.0 | 3.8 | 3.8 | Free from particles |
| 25° C. | 12 weeks | nd | 1.3 | 98.4 | 0.3 | 54.9 | 41.3 | 3.8 | 3.5 | Practically free from |
| | | | | | | | | | | particles |
| 40° C. | 12 weeks | nd | 1.4 | 92.9 | 5.7 | 85.4 | 11.7 | 3.0 | 4.2 | Free from particles |
| 2-8° C. | 24 weeks | nd | 1.4 | 98.5 | 0.2 | 50.2 | 46.2 | 3.6 | 3.5 | Free from particles |
| 25° C. | 24 weeks | nd | 1.3 | 98.2 | 0.4 | 61.5 | 35.2 | 3.3 | 3.4 | Free from particles |
| 40° C. | 24 weeks | nd | 2.1 | 88.6 | 9.3 | nd | nd | nd | 3.8 | Free from particles |
| 2-8° C. | 36 weeks | nd | 1.3 | 98.5 | 0.2 | 50.0 | 46.6 | 3.4 | 3.6 | Free from particles |
| 25° C. | 36 weeks | nd | 1.3 | 98.0 | 0.7 | 66.2 | 30.8 | 3.0 | 3.4 | Free from particles |
| 2-8° C. | 55 weeks | nd | 1.3 | 98.5 | 0.2 | 50.2 | 46.2 | 3.6 | 3.6 | Free from particles |
| 25° C. | 55 weeks | nd | 1.5 | 97.7 | 0.9 | 71.8 | 25.0 | 3.1 | 3.8 | Free from particles |

Formulation L is a liquid formulation with the composition 25 mg/ml
hu-ICR62 IgG1 anti-EGFR mAb, 20 mM L-histidine pH 5.5, 240 mM
sucrose, 0.02% polysorbate 80.

| | | | | | | | | | | |
|---|---|---|---|---|---|---|---|---|---|---|
| — | Initial | 24.2 | 1.3 | 98.5 | 0.1 | 49.6 | 47.2 | 3.2 | 3.6 | Free from particles |
| Shaking 5° C. | 1 week | 24.1 | 1.3 | 98.5 | 0.2 | 49.5 | 47.0 | 3.5 | 3.8 | Practically free from |
| | | | | | | | | | | particles |
| Shaking 25° C. | 1 week | 24.2 | 1.3 | 98.5 | 0.2 | 50.0 | 46.4 | 3.6 | 3.5 | Practically free from |
| | | | | | | | | | | particles |
| Freezing/ | (5 cycles) | 24.4 | 1.3 | 98.5 | 0.2 | 49.5 | 47.0 | 3.5 | 3.6 | Practically free from |
| Thawing | | | | | | | | | | particles |
| 2-8° C. | 4 weeks | nd | nd | nd | nd | nd | nd | nd | nd | nd |
| 25° C. | 4 weeks | nd | 1.3 | 98.5 | 0.2 | 51.5 | 44.9 | 3.6 | 3.6 | Free from particles |
| 40° C. | 4 weeks | nd | 1.1 | 98.2 | 0.7 | 69.5 | 27.3 | 3.3 | 3.8 | Free from particles |
| 2-8° C. | 12 weeks | nd | 1.3 | 98.5 | 0.2 | 49.3 | 46.9 | 3.8 | 3.8 | Free from particles |
| 25° C. | 12 weeks | nd | 1.3 | 98.4 | 0.3 | 54.8 | 41.4 | 3.8 | 3.9 | Practically free from |
| | | | | | | | | | | particles |
| 40° C. | 12 weeks | nd | 1.5 | 92.7 | 5.8 | 86.0 | 11.1 | 2.9 | 4.0 | Free from particles |
| 2-8° C. | 24 weeks | nd | 1.4 | 98.5 | 0.2 | 50.0 | 46.4 | 3.6 | 3.5 | Free from particles |
| 25° C. | 24 weeks | nd | 1.4 | 98.2 | 0.5 | 61.6 | 35.1 | 3.3 | 3.5 | Free from particles |
| 40° C. | 24 weeks | nd | 2.5 | 88.1 | 9.4 | nd | nd | nd | 3.9 | Free from particles |
| 2-8° C. | 36 weeks | nd | 1.3 | 98.5 | 0.2 | 50.1 | 46.4 | 3.5 | 3.4 | Practically free from |
| | | | | | | | | | | particles |
| 25° C. | 36 weeks | nd | 1.3 | 98.0 | 0.7 | 65.9 | 30.8 | 3.3 | 3.3 | Free from particles |
| 2-8° C. | 55 weeks | nd | 1.3 | 98.5 | 0.2 | 50.5 | 46.0 | 3.4 | 3.5 | Free from particles |
| 25° C. | 55 weeks | nd | 1.4 | 97.8 | 0.9 | 71.8 | 25.0 | 2.0 | 3.7 | Free from particles |

We claim:

1. A pharmaceutical formulation comprising:
1 to 200 mg/ml of an IgG-class anti-EGFR antibody;
1 to 100 mM of a buffering agent;
0.001 to 1% (w/v) of a surfactant;
1 to 500 mM of at least one stabilizer
at a pH in the range of from 4.0 to 7.0.

2. The formulation according to claim 1, wherein the IgG-class anti-EGFR antibody concentration is in the range of 1 to 100 mg/ml.

3. The formulation according to claim 1, wherein the buffering agent is a histidine buffer or an acetate buffer.

4. The formulation according to claim 1, wherein the buffering agent is at a concentration of 10 to 50 mM.

5. The formulation according to claim 1, wherein the buffering agent provides a pH of 5.0 to 6.0.

6. The formulation according to claim 1, wherein the surfactant is a polysorbate.

7. The formulation according to claim 1, wherein the surfactant is at a concentration of 0.01 to 0.1% (w/v).

8. The formulation according to claim 1, wherein at least one stabilizer is selected from the group of salts, saccharides, and amino acids.

9. The formulation according to claim 1, wherein the at least one stabilizer is at a concentration of 120 to 300 mM.

10. The formulation according to claim 1, comprising a first stabilizer selected from the group of salts, saccharides and amino acids, and methionine as a second stabilizer.

11. The formulation according to claim 10, wherein the first stabilizer is at a concentration of 120 to 300 mM, and the second stabilizer methionine is at a concentration of 5 to 25 mM.

12. The formulation according to claim 1, which comprises:
10 to 50 mg/ml of an IgG-class anti-EGFR antibody;
15 to 30 mM of a buffering agent, selected from L-histidine and sodium acetate;
0.02 to 0.05% (w/v) of a surfactant, selected from polysorbate 20 and polysorbate 80;
120 to 300 mM of at least one stabilizer, selected from trehalose dihydrate, sucrose, arginine hydrochloride and sodium chloride;
optionally 5 to 25 mM of methionine as a second stabilizer;
at a pH of 5.5±0.3.

13. The formulation according to claim 1, which comprises:
10 to 50 mg/ml IgG-class anti-EGFR antibody, 20 mM L-histidine, 240 mM trehalose dihydrate, 0.02 to 0.03% (w/v) polysorbate 80, at pH 5.5; or
10 to 50 mg/ml IgG-class anti-EGFR antibody, 20 mM L-histidine, 155 mM arginine hydrochloride, 0.02% (w/v) polysorbate 80, at pH 5.5; or
10 to 50 mg/ml IgG-class anti-EGFR antibody, 20 mM L-histidine, 240 mM trehalose dihydrate, 0.02 to 0.03% (w/v) polysorbate 80, 10 mM methionine at pH 5.5; or
10 to 50 mg/ml IgG-class anti-EGFR antibody, 20 mM L-histidine, 240 mM sucrose, 0.02 to 0.03% (w/v) polysorbate 80, at pH 5.5. or
10 to 50 mg/ml IgG-class anti-EGFR antibody, 20 mM L-histidine, 240 mM sucrose, 0.02 to 0.03% (w/v) polysorbate 80, 10 mM methionine, at pH 5.5.

14. The formulation according to claim 1, which comprises
20 to 50 mg/ml of hu-ICR62 IgG1 anti-EGFR mAb;
20 mM L-histidine;
0.02 to 0.03% (w/v) polysorbate 80;
240 mM of a first stabilizer, wherein said first stabilizer is a saccharide selected from trehalose dihydrate and sucrose;
10 mM of methionine as a second stabilizer;
at a pH of 5.5±0.3.

15. The formulation according to claim 1, wherein the IgG-class anti-EGFR antibody is a humanized antibody and comprises
a) in the heavy chain variable domain a CDR1 of SEQ ID NO:1, a CDR2 of SEQ ID NO:16 and a CDR3 of SEQ ID NO:31, and
b) in the light chain variable domain a CDR1 of SEQ ID NO:33, a CDR2 of SEQ ID NO:34 and a CDR3 of SEQ ID No:35.

16. The formulation according to claim 1, wherein the IgG-class anti-EGFR antibody is hu-ICR62 IgG1 anti-EGFR mAb.

17. The formulation according to claim 1, wherein the IgG-class anti-EGFR antibody has been glycoengineered to have an increased proportion of non-fucosylated oligosaccharides it its Fc region, as compared to the non-glycoengineered antibody.

18. The formulation according to claim 1, which is in a liquid form, in a lyophilized form or in a liquid form reconstituted from a lyophilized form.

19. Use of a formulation according to claim 1 for the preparation of a medicament useful for treating cancer.


SEQUENCE LISTING (40 sequences)

SEQ ID NO: 1 - protein, 5 aa, Artificial Sequence
  Heavy Chain CDR1 Kabat
DYKIH

SEQ ID NO: 2 - protein, 5 aa, Artificial Sequence
  Heavy Chain CDR1 Kabat
DYAIS

SEQ ID NO: 3 - protein, 5 aa, Artificial Sequence
  Heavy Chain CDR1 Kabat
DYYMH

SEQ ID NO: 4 - protein, 5 aa, Artificial Sequence
  Heavy Chain CDR1 Kabat
DYKIS

SEQ ID NO: 5 - protein, 7 aa, Artificial Sequence
  Heavy Chain CDR1 Chothia
GFTFTDY

SEQ ID NO: 6 - protein, 7 aa, Artificial Sequence
  Heavy Chain CDR1 Chothia
GYTFTDY

SEQ ID NO: 7 - protein, 7 aa, Artificial Sequence
  Heavy Chain CDR1 Chothia
GYSFTDY

SEQ ID NO: 8 - protein, 10 aa, Artificial Sequence
  Heavy Chain CDR1 AbM
GFTFTDYKIH

SEQ ID NO: 9 - protein, 10 aa, Artificial Sequence
  Heavy Chain CDR1 AbM
GFTFTDYAIS

SEQ ID NO: 10 - protein, 10 aa, Artificial Sequence
  Heavy Chain CDR1 AbM
GFTFTDYYMH

SEQ ID NO: 11 - protein, 10 aa, Artificial Sequence
  Heavy Chain CDR1 AbM
GYTFTDYYMH

SEQ ID NO: 12 - protein, 10 aa, Artificial Sequence
  Heavy Chain CDR1 AbM
GYSFTDYKIH

SEQ ID NO: 13 - protein, 10 aa, Artificial Sequence
  Heavy Chain CDR1 AbM
GFTFTDYKIS

SEQ ID NO: 14 - protein, 17 aa, Artificial Sequence
  Heavy Chain CDR2 Kabat
YFNPNSGYSTYNEKFKS

SEQ ID NO: 15 - protein, 17 aa, Artificial Sequence
  Heavy Chain CDR2 Kabat
GINPNSGYSTYAQKFQG

SEQ ID NO: 16 - protein, 17 aa, Artificial Sequence
  Heavy Chain CDR2 Kabat
YFNPNSGYSTYAQKFQG

SEQ ID NO: 17 - protein, 17 aa, Artificial Sequence
  Heavy Chain CDR2 Kabat
WINPNSGYSTYAQKFQG

SEQ ID NO: 18 - protein, 17 aa, Artificial Sequence
  Heavy Chain CDR2 Kabat
WINPNSGYSTYSPSFQG

SEQ ID NO: 19 - protein, 17 aa, Artificial Sequence
  Heavy Chain CDR2 Kabat
WINPNSGYSTYNEKFQG

SEQ ID NO: 20 - protein, 17 aa, Artificial Sequence
  Heavy Chain CDR2 Kabat
YFNPNSGYSNYAQKFQG

SEQ ID NO: 21 - protein, 17 aa, Artificial Sequence
  Heavy Chain CDR2 Kabat
YFNPNSGYATYAQKFQG

SEQ ID NO: 22 - protein, 17 aa, Artificial Sequence
  Heavy Chain CDR2 Kabat
YFNPNSGYSTYSPSFQG

SEQ ID NO: 23 - protein, 8 aa, Artificial Sequence
  Heavy Chain CDR2 Chothia
NPNSGYST

SEQ ID NO: 24 - protein, 8 aa, Artificial Sequence
  Heavy Chain CDR2 Chothia
NPNSGYSN

SEQ ID NO: 25 - protein, 8 aa, Artificial Sequence
  Heavy Chain CDR2 Chothia
NPNSGYAT

SEQ ID NO: 26 - protein, 10 aa, Artificial Sequence
  Heavy Chain CDR2 AbM
YFNPNSGYST

SEQ ID NO: 27 - protein, 10 aa, Artificial Sequence
  Heavy Chain CDR2 AbM
GINPNSGYST

SEQ ID NO: 28 - protein, 10 aa, Artificial Sequence
  Heavy Chain CDR2 AbM
WINPNSGYST

SEQ ID NO: 29 - protein, 10 aa, Artificial Sequence
  Heavy Chain CDR2 AbM
YFNPNSGYSN

SEQ ID NO: 30 - protein, 10 aa, Artificial Sequence
  Heavy Chain CDR2 AbM
YFNPNSGYAT

SEQ ID NO: 31 - protein, 11 aa, Artificial Sequence
  Heavy Chain CDR3 Kabat Chothia AbM
LSPGGYYVMDA

SEQ ID NO: 32 - protein, 11 aa, Artificial Sequence
  Light Chain CDR1 Kabat
KASQNINNYLN

SEQ ID NO: 33 - protein, 11 aa, Artificial Sequence
  Light Chain CDR1 Kabat
RASQGINNYLN

SEQ ID NO: 34 - protein, 7 aa, Artificial Sequence
  Light Chain CDR2 Kabat
NTNNLQT

SEQ ID NO: 35 - protein, 8 aa, Artificial Sequence
  Light Chain CDR3 Kabat
LQHNSFPT

SEQ ID NO: 36 - protein, 120 aa, Rattus norvegicus
  MISC_FEATURE: ICR62 VH
QVNLLQSGAALVKPGASVKLSCKGSGFTFTDYKIHWVKQSHGKSLEWIGYFNPNSGYSTYNEKFKSKATLTADKSTDTAYMELTSLTSEDSATYYCTRLSPGGYYVMDAWGQGASVTVSS

SEQ ID NO: 37 - protein, 108 aa, Rattus norvegicus
  MISC_FEATURE: ICR62 VL
DIQMTQSPSFLSASVGDRVTINCKASQNINNYLNWYQQKLGEAPKRLIYNTNNLQTGIPSRFSGSGSGTDYTLTISSLQPEDFATYFCLQHNSFPTFGAGTKLELKRT

SEQ ID NO: 38 - protein, 120 aa, Artificial Sequence [sequence omitted]

SEQ ID NO: 39 - protein, 108 aa, Artificial Sequence
  I-KC VL construct
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRT

SEQ ID NO: 40 - protein, 328 aa, Homo sapiens
  MISC_FEATURE: Ig gamma-1 CH region
TKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK